(19) United States
(12) Patent Application Publication
(10) Pub. No.: US 2005/0229331 A1
(43) Pub. Date: Oct. 20, 2005

(54) Composition for dyeing keratin fibers, comprising at least one alcohol oxidase enzyme and at least one polymer comprising at least one sugar unit, and process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,271

(22) Filed: Jan. 28, 2005

Related U.S. Application Data
(60) Provisional application No. 60/545,133, filed on Feb. 18, 2004.

(30) Foreign Application Priority Data
Jan. 28, 2004 (FR) 0400777

(51) Int. Cl. A61K 7/13

(52) U.S. Cl. 8/405

(57) ABSTRACT

Disclosed herein is a composition for dyeing keratin fibers, for example, human keratin fibers such as the hair, comprising, in a medium suitable for dyeing, at least one oxidation dye precursor, at least one alcohol oxidase enzyme, at least one substrate for the enzyme, and at least one polymer comprising at least one sugar unit. Further disclosed herein are a process for dyeing keratin fibers, comprising applying this composition to the keratin fibers, and a multi-compartment device comprising the composition.

This application claims benefit of U.S. Provisional Application No. 60/545,133, filed Feb. 18, 2004.

The present disclosure relates to a composition for dyeing keratin fibers, for example, human keratin fibers such as the hair, comprising, in a medium suitable for dyeing, at least one oxidation dye precursor, at least one alcohol oxidase enzyme, at least one substrate for the enzyme, and at least one polymer comprising at least one sugar unit.

It is known practice to dye keratin fibers, such as human hair, with dye compositions comprising oxidation dye precursors, such as ortho- or para-phenylenediamines, ortho- or para-aminophenols, and heterocyclic compounds, which are generally referred to as oxidation bases. These oxidation bases, are colorless or weakly colored compounds which, when combined with oxidizing products, may give rise to colored compounds by a process of oxidative condensation.

It is also known that the shades obtained with these oxidation bases may be varied by combining them with couplers or coloration modifiers, which may be chosen, for example, from aromatic meta-diamines, meta-aminophenols, meta-diphenols and certain heterocyclic compounds such as indole compounds.

The variety of molecules used as oxidation bases and couplers makes it possible to obtain a wide range of colors.

The “permanent” coloration obtained by these oxidation dyes should satisfy a certain number of requirements. For example, it should have no toxicological drawbacks and it should allow shades of the desired intensity to be obtained and have good resistance to external agents such as light, bad weather, washing, permanent waving, perspiration and rubbing.

The dyes should also allow white hairs to be covered and they should be as unselective as possible, i.e., they should allow the smallest possible differences in coloration to be produced over the entire length of the same keratin fiber, which is generally differently sensitized (i.e. damaged) between its end and its root.

The dyeing is generally performed in a strongly alkaline medium, in the presence of hydrogen peroxide. However, the use of alkaline media in the presence of hydrogen peroxide may have the drawback of causing considerable degradation of the fibers, and bleaching of keratin fibers, which is not always desirable.

The oxidation dyeing of keratin fibers may also be performed using oxidizing systems other than hydrogen peroxide, such as enzymatic systems. For example, French patent application FR 2 769 219 describes the use of a uricase enzyme and its uric acid substrate in oxidation dyeing to dye keratin fibers. European patent application EP-A-0 310 675 describes the use of oxidation dye precursors of benzenic type in combination with enzymes such as pyranose oxidase and glucose oxidase. More recently, French patent application FR 2 833 492 describes the use of the alcohol oxidase enzyme as sole enzyme in an oxidation dye composition for dyeing keratin fibers.

Disclosed herein are novel thickened and stable compositions for dyeing keratin fibers by oxidation dyeing, using an oxidizing system other than hydrogen peroxide.

As used herein, the term “thickened composition” means a composition that can be applied to the area to be dyed without running outside this area.

For example, the viscosity of the compositions as disclosed herein is greater than 200 cp such as greater than 500 cp, measured at 25° C. using a Rheomat RM 180 rheometer at a shear rate of 200 s⁻¹.

The present inventor has now discovered, advantageously and unexpectedly, that it is possible to obtain the thickened and stable composition by using at least one oxidation dye precursor, at least one alcohol oxidase enzyme, at least one substrate for the enzyme, and at least one polymer comprising at least one sugar unit in a composition for dyeing keratin fibers, for example, human keratin fibers such as the hair.

The composition as disclosed herein makes it possible to obtain, for example, homogeneous formulations which, once applied, respect the nature of the keratin fibers and do not have the solubilization and crystallization problems encountered with, for example, the uric acid/uricase system. The present inventor has also noted that the stability of the composition as disclosed herein, for example, the stability of the alcohol oxidase enzyme of this composition, may be improved.

The compositions as disclosed herein may also have the advantage that they can lead to the production of dyeing results with strong, unselective and fast colors, and are capable of generating varied shades of intense and uniform color, without any significant degradation of the hair. In addition, it has been noted that the use of such a composition may improve the hold of permanent-waved hair and reduce the porosity of the hair.

The present inventor has also observed that, during the use of the composition as disclosed herein, the uptake of the dye onto the fibers is not halted, which is generally the case during the use of dye compositions comprising conventional thickeners, surfactants and solvents.

When the uptake of the dye onto the fibers is halted, then producing a strong shade may require the use of a larger amount of dye and also a larger amount of solvent and/or surfactant to dissolve this dye.

Other characteristics, aspects, subjects and advantages of the present disclosure will emerge even more clearly on reading the description and the examples that follow.

The at least one polymer comprising at least one sugar unit may be associative or non-associative.

As used herein, the term “associative polymer” means a polymer comprising in its structure at least one C₈-C₃₀ side or end fatty chain. These associative polymers are capable, in aqueous medium, of reversibly combining together or with other molecules such as surfactants to lead to increased thickening of the medium.

Among the polymers comprising at least one sugar unit that may be used in the compositions as disclosed herein, mention may be made, for example, of:
nonionic guar gums;
biopolysaccharide gums of microbial origin such as scleroglucan gum and xanthan gum;
gums derived from plant exudates, such as gum arabic, ghatti gum, karaya gum, gum tragacanth, carrageenan, agar and carob gum;
pectins;
alginates;
starches;
cellulose derivatives chosen from hydroxyalkylcelluloses and carboxyalkylcelluloses, cationic celluloses, cationic hydroxyethylcelluloses, nonionic cellulose derivatives and modified cellulose derivatives.

As used herein, the term “sugar unit” means a monosaccharide portion (i.e. monosaccharide or oside or simple sugar) or an oligosaccharide portion (short chains formed from the linking of monosaccharide units, which may be identical or different) or a polysaccharide portion (i.e., long chains comprising monosaccharide units, which may be identical or different, i.e., polyholosides or polyosides (homopolyosides or heteropolyosides)). The saccharide units can also be substituted with at least one radical chosen from alkyl, hydroxyalkyl, alkoxy, acyloxy and carboxyl radicals.

The nonionic guar gums can be modified or unmodified.

The unmodified guar gums are, for example, the products sold under the name Vidogum GH 175 by the company Unipectine and under the name Jaguar C by the company Meyhall.

As disclosed herein, nonionic guar gums modified with at least one group chosen from C₁-C₆ hydroxyalkyl groups are, for example, used.

Among the hydroxyalkyl groups that may be mentioned, examples include hydroxymethyl, hydroxyethyl, hydroxypropyl and hydroxybutyl groups.

These guar gums are well known in the art and can be prepared, for example, by reacting the corresponding alkene oxides, for example, propylene oxides, with the guar gum so as to obtain a guar gum modified with hydroxypropyl groups.

The degree of hydroxyalkylation, which corresponds to the number of alkylene oxide molecules consumed by the number of free hydroxyl functional groups present on the guar gum, ranges, for example, from 0.4 to 1.2.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Rhô ne-Poulenc (Meyhall) or under the name Galactasol 4H4FD2 by the company Aqualon.

The biopolysaccharide gums of microbial origin, such as the scleroglucan or xanthan gums, the gums derived from plant exudates such as gum arabic, ghatti gum, karaya gum, gum tragacanth, carrageenan gum, agar gum and carob gum, the hydroxyalkylcelluloses and carboxymethylcelluloses, pectins, alginates and starches are well known to those skilled in the art and are described, for example, in the book by Robert L. Davidson entitled “Handbook of Water soluble gums and resins” published by McGraw Hill Book Company (1980).

Among these gums, examples include scleroglucans, such as those sold under the name Actigum CS by the company Sanofi Bio Industries, for example, Actigum CS 11, and under the name Amigel by the company Alban Muller International. Other scleroglucans, such as the one treated with glyoxal disclosed in French patent application No. 2 633 940, can also be used.

The xanthan gums are chosen, for example, from the products sold under the names Keltrol, Keltrol T, Keltrof TF, Keltrol BT, Keltrol RD and Keltrol CG by the company Nutrasweet Kelco, or under the names Rhodicare S and Rhodicare H by the company Rhodia Chimie.

The hydroxyalkylcelluloses may be chosen, for example, from hydroxyethylcelluloses, such as those sold under the names Cellosize QP3L, Cellosize QP4400H, Cellosize QP30000H, Cellosize HEC30000A and Cellosize Polymer PCG10 by the company Amerchol, or Natrosol 250HHR, Natrosol 250MR, Natrosol 250M, Natrosol 250HHXR, Natrosol 250HHX, Natrosol 250HR and Natrosol HX by the company Hercules, or Tylose H1000 by the company Hoechst.

The hydroxyalkylcelluloses may also be chosen, for example, from hydroxypropylcelluloses such as the products sold under the names Klucel EF, Klucel H, Klucel LHF, Klucel MF and Klucel G by the company Aqualon.

Among the carboxyalkylcelluloses, examples include carboxymethylcellulose, such as the products sold under the names Blanose 7M8/SF, Blanose Raffinée 7M, Blanose 7LF, Blanose 7MF, Blanose 9M31F, Blanose 12M31XP, Blanose 12M31P, Blanose 9M31XF, Blanose 7H, Blanose 7M31 and Blanose 7H3SXF by the company Aqualon, or Aquasorb A500 and Ambergum 1221 by the company Hercules, or Cellogen HP810A and Cellogen HP6HS9 by the company Montello, or Primellose by the company Avebe.

The cationic celluloses may be chosen, for example, from quaternized cationic celluloses modified with groups comprising at least one hydrophobic chain, such as alkyl, arylalkyl or alkylaryl groups comprising at least 8 carbon atoms, or mixtures thereof.

The cationic hydroxyethylcelluloses may be chosen, for example, from quaternized cationic hydroxyethylcelluloses modified with groups comprising at least one hydrophobic chain, such as alkyl, arylalkyl or alkylaryl groups comprising at least 8 carbon atoms, or mixtures thereof.

The alkyl radicals borne by the above quaternized celluloses or hydroxyethylcelluloses comprise, for example, from 8 to 30 carbon atoms. The aryl radicals are chosen, for example, from phenyl, benzyl, naphthyl and anthryl groups.

Examples of quaternized alkylhydroxyethylcelluloses comprising C₈-C₃₀ hydrophobic chains that may be mentioned include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol, and the products Crodacel QM®, Crodacel QL® (C₁₂ alkyl) and Crodacel QS® (C₁₈ alkyl) sold by the company Croda.

The nonionic cellulose derivatives may be chosen, for example, from hydroxyethylcelluloses modified with groups comprising at least one hydrophobic chain, such as alkyl, arylalkyl or alkylaryl groups, or mixtures thereof, and in which the alkyl groups are chosen, for example, from C₈-C₂₂ alkyl groups, for instance the product Natrosol Plus Grade 330 CS® (C₁₆ alkyls) sold by the company Aqualon, or the product Bermocoll EHM 100® sold by the company Berol Nobel.

Another family of cellulose-based polymers that may be used is, for example, cellulose derivatives modified with polyalkylene glycol alkylphenol ether groups, such as the product Amercell Polymer HM-1500® sold by the company Amerchol.

The at least one polymer comprising at least one sugar unit is present in an amount ranging, for example, from 0.01% to 10% by weight, such as from 0.1% to 5% by weight, relative to the total weight of the dye composition.

As disclosed herein, the at least one alcohol oxidase enzyme used in the dye composition as disclosed herein belongs to the class E.C.1.1.3 of the enzyme nomenclature (see Enzyme Nomenclature, Academic Press Inc; 1992).

The enzymes are chosen, for example, from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

In one embodiment, the enzyme used in the composition as disclosed herein is a primary alcohol oxidase (EC1.1.3.13).

Alcohol oxidase enzymes form, for example, a class of two-electron oxidoreductase enzymes.

The at least one alcohol oxidase enzyme used in the dye composition as disclosed herein may be derived from an extract of plants, of animals, of micro-organisms (bacterium, fungus, yeast, microalga or virus), of differentiated or undifferentiated cells, obtained in vivo or in vitro, unmodified or genetically modified, or synthetic (obtained by chemical or biotechnological synthesis).

By way of example, the at least one alcohol oxidase enzyme may be derived from one of the following species: Rhodococcus erythropolis, Pseudomonas pseudoalcaligenes which are bacteria, Aspergillus niger, Kamagataella pastoris, Phanerochaete chrysosporium, Polyporus obtusus, Hansenula polymorpha, Poria contigua, Penicillium simplicissimum, Pleurotus pulmonarius (fungi), Pichia sp. (pastoris, methanolica, angusta) and Candida sp. (boidinii, albicans, tropicalis) (yeasts), Pinus strobus, which is a species of plant origin, Gastropode mollusc and Manduca sexta, which are of animal origin.

In one embodiment, the enzyme used in the composition as disclosed herein is an oxidase alcohol derived from Pichia pastoris.

Generally, the at least one alcohol oxidase enzyme is present in an amount ranging, for example, from 0.05% to 20% by weight, such as from 0.1% to 10% by weight, and further such as from 0.5% to 8% by weight relative to the total weight of the composition.

The enzymatic activity of the at least one alcohol oxidase enzymes may be defined from the oxidation of the donor under aerobic conditions. One unit U corresponds to the amount of enzyme leading to the generation of 1 μmol of hydrogen peroxide per minute at a given pH and at a temperature of 25° C.

The amount of the at least one alcohol oxidase ranges, for example, from 10³ U to 10⁵ U, such as from 2×10³ U to 5×10⁴ U, per 100 g of the dye composition.

The at least one substrate for the enzyme is also known as donors for the enzyme.

The at least one substrate for the enzyme used in the compositions as disclosed herein is, for example, an alcohol chosen from primary alcohols, secondary alcohols, long-hydrocarbon-chain alcohols and aromatic alcohols. For example, donors for the primary alcohol oxidases that may be mentioned include primary alcohols comprising from 1 to 6 carbon atoms; donors for the aryl alcohol oxidases that may be mentioned include benzyl alcohol, 4-tert-butylbenzyl alcohol, 3-hydroxy-4-methoxybenzyl alcohol, veratryl alcohol, 4-methoxybenzyl alcohol and cinnamyl alcohol; 2,4-hexadien-1-ol may also be used as donor for the aryl alcohol oxidases.

According to another variant, the substrate for the enzyme is a compound bearing at least one alcohol functional group chosen from aliphatic and aromatic alcohol functional groups, suitable for reacting with the enzyme used. The compound bearing at least one alcohol functional group chosen from aliphatic and aromatic alcohol functional groups may, for example, be an oxidation dye precursor or a cosmetically acceptable adjuvant, for example, a polymer, a surfactant or a preserving agent bearing at least one alcohol functional group. In one embodiment, the substrate for the enzyme is an oxidation dye precursor bearing at least one alcohol function group chosen from aliphatic and aromatic alcohol functional groups. For example, N-(β-hydroxypropyl)-para-phenylenediamine, which bears a primary alcohol functional group, may serve as oxidation base and as substrate for the alcohol oxidase. Similarly, couplers, such as meta- or para-aminophenol, may fulfil the two functions. Such couplers are described hereinbelow. In this variant, the use of other substrates for the enzyme is optional.

Thus, disclosed herein is a composition for dyeing keratin fibers, for example, human keratin fibers such as the hair, comprising, in a medium suitable for dyeing, at least one oxidation dye precursor; at least one alcohol oxidase enzyme; at least one substrate, bearing at least one alcohol functional group, for the enzyme and at least one polymer comprising at least one sugar unit, wherein the at least one substrate may be totally or partially replaced by the at least one oxidation dye precursor in the case where the at least one oxidation dye precursor bears at least one alcohol functional group chosen from aliphatic and aromatic alcohol functional groups.

The use of the composition as disclosed herein makes it possible to reduce the risks associated with the handling of hydrogen peroxide. Furthermore, the concentration of preserving agents in the compositions as disclosed herein may be reduced by providing compounds comprising at least one alcohol functional group that also have preserving properties.

Generally, the at least one substrate for the enzyme is present in an amount ranging, for example, from 0.01% to 60% by weight, such as from 0.05% to 30% by weight, relative to the total weight of the composition.

The at least one oxidation dye precursor may be chosen, for example, from para-phenylenediamines, bis(phenyl)alkylenediamines, para-aminophenols, ortho-aminophenols and heterocyclic bases, and the addition salts thereof.

Among the para-phenylenediamines, mention may be made, for example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxy-propyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′aminophenyl)pyrrolidine, and the acid addition salts thereof.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the acid addition salts thereof may, for example, be used.

Among the bis(phenyl)alkylenediamines, mention may be made, for example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetra-methylened iamine, N,N′-bis(β-hydroxyethyl)-N,N′-bis(4-aminophenyl)tetra-methylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the acid addition salts thereof.

Among the para-aminophenols, mention may be made, for example, of para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethyl phenol, 4-amino-2-methoxymethyl phenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol and bis(5′-amino-2′-hydroxy)phenylmethane and the acid addition salts thereof.

Among the ortho-aminophenols, mention may be made, for example, of 2-aminophenol, 2-amino-5-methylphenol, 2-amino-6-methylphenol and 5-acetamido-2-aminophenol, and the acid addition salts thereof.

Among the heterocyclic bases, mention may be made, for example, of pyridine derivatives, pyrimidine derivatives and pyrazole derivatives.

Among the pyridine derivatives, mention may be made of the compounds described, for example, in British patents GB 1 026 978 and GB 1 153 196, such as 2,5-diaminopyridine, 2-(4-methoxyphenyl)amino-3-aminopyridine, and 3,4-diaminopyridine, and the acid addition salts thereof.

Other pyridine oxidation bases that are useful herein include, for example, the 3-aminopyrazolo[1,5-a]pyridine oxidation bases or the addition salts thereof described, for example, in French patent application FR 2 801 308. Examples that may be mentioned include pyrazolo[1,5-a]pyrid-3-ylamine; 2-acetylaminopyrazolo[1,5-a]pyrid-3-ylamine; 2-morpholin-4ylprazolo[1,5]pyrid-3-ylamine; 3-aminopyrazolo[1,5-a]pyridin-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl) ethanol; (3-aminopyrazolo[1,5-a]pyrid-2-yl)methanol; 3,6-diaminopyrazolo[1,5-a]pyridine; 3,4-diaminopyrazolo[1,5-a]pyrid-3,7-diamine; 7-morpholin-4-ylpyrazolo-[1,5-a]pyrid-3-ylamine; pyrazolo[1,5-a]pyrid-3,5-diamine; 5-morpholin-4-ylpyrazolo[1,5-a]pyrid-3-ylamine; 2-[(3-aminopyrazolo[1,5-a]pyrid-5-yl)-(2-hydroxyethyl)amino]ethanol; 2-[(3-aminopyrazolo[1,5-a]pyrid-7-yl)-(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyridine-6-ol; 3-aminopyrazolo[1,5-a]pyrid-7-ol; and the acid addition salts thereof.

Among the pyrimidine derivatives, mention may be made of the compounds described, for example, in patents DE 2 359 399; JP 88-169 571; JP 05 163 124; EP 0 770 375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine and the addition salts thereof and the tautomeric forms thereof, when a tautomeric equilibrium exists.

Among the pyrazole derivatives, mention may be made of the compounds described, for example, in German patents DE 3 843 892, DE 4 133 957 and patent applications WO 94/08969, WO 94/08970, FR-A-2,733,749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy-methylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof.

Generally, the at least one oxidation dye precursor is present in an amount ranging, for example, from 0.0001% to 20% by weight, such as from 0.005% to 6% by weight, relative to the total weight of the composition.

The composition as disclosed herein may also comprise at least one coupler, chosen, for example, from meta-phenylenediamines, meta-aminophenols, meta-diphenols, naphthalene-based couplers and heterocyclic couplers, and the addition salts thereof.

Examples of the at least one coupler that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureidoaniline, 3-ureido-1-dimethylaminobenzene, sesamol, 1-β-hydroxyethylamino-3,4-methylenedioxy-benzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

Generally, the at least one coupler is present in an amount ranging, for example, from 0.0001% to 20% by weight, such as from 0.005% to 6% by weight, relative to the total weight of the composition.

In general, the acid addition salts that may be used for the oxidation dye precursors and couplers are chosen, for example, from hydrochlorides, hydrobromides, sulfates, citrates, succinates, tartrates, lactates, tosylates, benzenesulfonates, phosphates and acetates.

The base addition salts that may be used herein are chosen, for example, from the addition salts with sodium hydroxide, potassium hydroxide, ammonia, amines and alkanolamines.

The dye composition as disclosed herein may also comprise at least one direct dye, which may be chosen, for example, from neutral, acidic or cationic nitrobenzene dyes, neutral, acidic or cationic azo direct dyes, neutral, acidic or cationic quinone such as anthraquinone direct dyes, azine direct dyes, methine, azomethine, triarylmethane and indoamine direct dyes, and natural direct dyes. In one embodiment, the at least one direct dye is chosen from cationic direct dyes and natural direct dyes.

Among the cationic direct dyes that may be used herein, mention may be made, for example, of the cationic azo direct dyes described in patent applications WO 95/15144, WO 95/01772 and EP 714 954.

Among these compounds, mention may be made, for example, of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]pyridinium methyl sulfate.

Among the natural direct dyes that may be used herein, mention may be made, for example, of lawsone, juglone, alizarin, purpurin, carminic acid, kermesic acid, purpurogallin, protocatechaldehyde, indigo, isatin, curcumin, spinulosin, and apigenidin. It is also possible to use extracts or decoctions comprising these natural dyes such as henna-based poultices or extracts.

The at least one direct dye is present in an amount ranging, for example, from 0.001% to 20% by weight, such as from 0.005% to 10% by weight, relative to the total weight of the composition.

The dye composition as disclosed herein may also comprise at least one adjuvant chosen from various cosmetically acceptable adjuvants other than polymers containing sugar units. These additional agents are conventionally used in compositions for dyeing the hair, and include, for example, antioxidants, penetrating agents, sequestering agents, fragrances, buffers, dispersants, surfactants, conditioners such as volatile or non-volatile, modified or unmodified silicones, cationic polymers, cations, film-forming agents, ceramides, preserving agents, opacifiers, vitamins and provitamins.

The at least one adjuvant is present in an amount ranging, for example, from 0.01% to 20% by weight relative to the weight of the composition.

The person skilled in the art will take care to select this or these optional additional compound(s) such that the advantageous properties intrinsically associated with the oxidation dye composition as disclosed herein are not, or are not substantially, adversely affected by the envisaged addition(s).

The medium suitable for dyeing, also known as the dye support, generally comprises water or a mixture of water and at least one organic solvent to dissolve the compounds that would not be sufficiently water-soluble. As appropriate, the at least one organic solvent may be a substrate of the enzyme such as ethanol or isopropanol. It may also be a compound that is a non-substrate for the enzyme, such as polyol ethers, for instance 2-butoxyethanol, propylene glycol, propylene glycol monomethyl ether and diethylene glycol monomethyl ether and monoethyl ether or phenoxyethanol, and mixtures thereof.

The at least one organic solvent may be present in an amount ranging, for example, from 1% to 40% by weight, such as from 5% to 30% by weight, relative to the total weight of the dye composition.

The pH of the dye composition as disclosed herein ranges, for example from 6 to 11, such as from 7 to 10. The pH may be adjusted for the desired value using acidifying or basifying agents commonly used in the dyeing of keratin fibers, or alternatively using standard buffer systems.

Among the acidifying agents that may be mentioned, examples include mineral or organic acids, for instance hydrochloric acid, orthophosphoric acid, sulfuric acid, carboxylic acids, for instance acetic acid, tartaric acid, citric acid and lactic acid, and sulfonic acids.

Among the basifying agents that may be mentioned, examples include aqueous ammonia, alkyl metal carbonates, alkanolamines such as monoethanolamine, diethanolamine and triethanolamine and derivatives thereof, sodium hydroxide, potassium hydroxide and the compounds of formula (III) below: 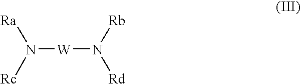
wherein W is a propylene residue optionally substituted with at least one radical chosen from hydroxyl and C₁-C₄ alkyl radicals; Ra, Rb, Rc and Rd, which may be identical or different, are each chosen from a hydrogen atom and C₁-C₄ alkyl and C₁-C₄ hydroxyalkyl radicals.

The dye composition may be in various forms, such as in the form of thickened liquids, creams or gels, or in any other form that is suitable for dyeing keratin fibers, such as human hair.

When the at least one oxidation dye precursor and the at least one alcohol oxidase are present in the same ready-to-use composition, the composition is, for example, free of oxygen gas, so as to avoid any premature oxidation of the oxidation dye precursor.

Further disclosed herein is a process for dyeing keratin fibers, for example, human keratin fibers such as the hair, comprising applying to the keratin fibers at least one dye composition as disclosed herein for a period of time sufficient to develop the desired coloration.

The color is then revealed by bringing together the alcohol oxidase enzyme and its substrate in the presence of oxygen.

The composition is applied to the keratin fibers. After leaving it to act for 3 to 60 minutes such as from 5 to 40 minutes, the keratin fibers are rinsed, washed with shampoo, rinsed again and then dried.

When the dye composition is a composition in ready-to-use form, it comprises, in a medium suitable for dyeing keratin fibers, at least one oxidation dye precursor, at least one alcohol oxidase enzyme, at least one substrate for the enzyme, and at least one polymer comprising at least one sugar unit, and the mixture is then stored in anaerobic form, free of oxygen gas.

According to one variant, the process comprises a preliminary operation comprising separately storing, on the one hand, a composition (A) comprising, in a medium suitable for dyeing keratin fibers, at least one oxidation dye precursor, and, on the other hand, a composition (B) comprising, in a medium suitable for dyeing keratin fibers, at least one alcohol oxidase enzyme, wherein at least one of the composition (A) and the composition (B) comprises at least one substrate for the enzyme and at least one polymer comprising at least one sugar unit, and then mixing together the compositions (A) and (B) at the time of use before applying this mixture to the keratin fibers.

According to another variant, the process comprises a preliminary operation comprising separately storing, on the one hand, a composition (A) comprising, in a medium suitable for dyeing keratin fibers, at least one oxidation dye precursor, at least one substrate for the enzyme, and at least one polymer comprising at least one sugar unit and, on the other hand, a composition (B) comprising, in a medium suitable for dyeing keratin fibers, at least one alcohol oxidase enzyme, and then mixing together the compositions (A) and (B) at the time of use before applying this mixture to the keratin fibers.

The color may be revealed at acidic, neutral or alkaline pH. In the case where the process is performed using a composition (A) comprising at least one oxidation dye precursor, at least one substrate for the enzyme and at least one polymer comprising at least one sugar unit and a composition (B) comprising at least one alcohol oxidase enzyme, the enzyme may be added to the final composition just at the time of use, or it may be used starting with the composition(B), applied simultaneously or sequentially to the composition (A).

In this case, the composition (B) (referred to as the oxidizing composition) may also comprise at least one adjuvant chosen from various adjuvants conventionally used in compositions for dyeing the hair as defined above.

The pH of the oxidizing composition (B) is such that, after mixing with the dye composition (A), the pH of the resulting composition applied to the keratin fibers ranges, for example, from 6 to 11, such as from 7 to 10. The pH may be adjusted to the desired value by acidifying or basifying agents commonly used in the dyeing of keratin fibers as defined above.

In one embodiment, the application of the composition as disclosed herein is carried out at a temperature ranging from the ambient temperature to 220° C., such as from the ambient temperature to 60° C.

Further disclosed herein is a multi-compartment device or dyeing “kit”, comprising a first compartment comprising the composition (A) as defined above and a second compartment comprising the composition (B) as defined above. This device may be equipped with an applicator for applying the desired mixture to the hair, such as the devices described in French patent FR-2 586 913.

The examples that follow serve to illustrate the invention without, however, being limiting in nature.

### EXAMPLES

The compositions of the following examples were prepared.

| Components | Example 1 | | Example 2 | | Example 3 | | Example 4 | |
|---|---|---|---|---|---|---|---|---|
| Hydroxyethylcellulose | 0.2 | g | | | | | | |
| sold under the name | | | | | | | | |
| Natrosol 250 HHR by | | | | | | | | |
| the company Aqualon | | | | | | | | |
| Carboxymethylcellulose | | | 0.2 | g | | | | |
| sold under the name | | | | | | | | |
| Blanose 7M by the | | | | | | | | |
| company Aqualon | | | | | | | | |
| Guar gum sold under | | | | | 0.2 | g | | |
| the name Vidogum | | | | | | | | |
| GH175 by the company | | | | | | | | |
| Unipectine | | | | | | | | |
| Scleroglucan gum sold | | | | | | | 0.2 | g |
| under the name Amigel | | | | | | | | |
| by the company Alban | | | | | | | | |
| Muller International | | | | | | | | |
| Ethanol | 25 | g | 25 | g | 25 | g | 25 | g |
| para-Phenylenediamine | 3 × 10⁻³ | mol | 3 × 10⁻³ | mol | 3 × 10⁻³ | mol | 3 × 10⁻³ | mol |
| meta-Aminophenol | 3 × 10⁻³ | mol | 3 × 10⁻³ | mol | 3 × 10⁻³ | mol | 3 × 10⁻³ | mol |
| Alcohol oxidase | 20000 | units | 20000 | units | 20000 | units | 20000 | units |
| 2-Amino-2-methyl-1- | pH 7 | | pH 7 | | pH 7 | | pH 7 | |
| propanol qs | | | | | | | | |
| Distilled water qs | 100 | g | 100 | g | 100 | g | 100 | g |

The alcohol oxidase used was the product sold by the company Biozyme Laboratories in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above compositions were applied to locks of natural grey permanent-waved hair comprising 90% white hairs, and left to act for 30 minutes. The bath ratio was set at 5 (the bath ratio is the ratio of the amount of the composition applied over the weight of the locks). The alcohol oxidase was added extemporaneously. The hair was then rinsed, washed with a standard shampoo and then dried.

The hair was dyed in shades of khaki green.

1. A composition for dyeing keratin fibers, comprising, in a medium suitable for dyeing, at least one oxidation dye precursor; at least one alcohol oxidase enzyme; at least one substrate bearing at least one alcohol functional group for said enzyme; and at least one polymer comprising at least one sugar unit, wherein said at least one substrate may be totally or partially replaced by the at least one oxidation dye precursor in the case where the at least one oxidation dye precursor bears at least one alcohol functional group chosen from aromatic and aliphatic alcohol functional groups.

2. The composition according to claim 1, wherein the keratin fibers are human hair.

3. The composition according to claim 1, wherein the at least one polymer comprising at least one sugar unit is associative.

4. The composition according to claim 1, wherein the at least one polymer comprising at least one sugar unit is non-associative.

5. The composition according to claim 1, wherein the at least one polymer comprising at least one sugar unit is chosen from nonionic guar gums, biopolysaccharide gums of microbial origin, gums derived from plant exudates, pectins, alginates, starches, cellulose derivatives chosen from hydroxyalkylcelluloses and carboxyalkylcelluloses, cationic celluloses, cationic hydroxyethylcelluloses, nonionic cellulose derivatives and modified cellulose derivatives.

6. The composition according to claim 5, wherein the biopolysaccharide gums of microbial origin are chosen from scleroglucan gum and xanthan gum.

7. The composition according to claim 5, wherein the gums derived from plant exudates are chosen from gum arabic, ghatti gum, karaya gum, gum tragacanth, carrageenan, agar and carob gum.

8. The composition according to claim 5, wherein the hydroxyalkylcelluloses are chosen from hydroxyethylcellulose and hydroxypropylcellulose.

9. The composition according to claim 5, wherein the nonionic guar gums are chosen from nonionic guar gums modified with at least one group chosen from C₁-C₆ hydroxyalkyl groups.

10. The composition according to claim 9, wherein at least one of the nonionic guar gums has a degree of hydroxyalkylation ranging from 0.4 to 1.2.

11. The composition according to claim 5, wherein the carboxyalkylcellulose is a carboxyethylcellulose.

12. The composition according to claim 5, the at least one polymer comprising at least one sugar unit is cationic and is chosen from celluloses and hydroxyethylcelluloses quaternized with at least one hydrophobic group.

13. The composition according to claim 5, wherein the at least one polymer comprising at least one sugar unit is nonionic and is chosen from hydroxyethylcelluloses modified with at least one hydrophobic group.

14. The composition according to claim 1, wherein the at least one polymer comprising at least one sugar unit is present in an amount ranging from 0.01% to 10% by weight relative to the total weight of the composition.

15. The composition according to claim 1, wherein the at least one alcohol oxidase enzyme is chosen from primary alcohol oxidases (EC 1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

16. The composition according to claim 15, wherein the at least one alcohol oxidase enzyme is derived from one of the following species: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia pastoris, Pichia methanolica, Pichia angusta, Candida boidinii, Candida albicans, Candida tropicalis, Pinus strobus, Gastropode mollusc, and Manduca sexta.

17. The composition according to claim 1, wherein the at least one alcohol oxidase enzyme is present in an amount ranging from 0.05% to 20% by weight relative to the total weight of the composition.

18. The composition according to claim 1, wherein the at least one alcohol oxidase enzyme is present in an amount ranging from 10³ U to 10⁵ U per 100 g of the composition.

19. The composition according to claim 1, wherein the at least one substrate for the enzyme is an alcohol chosen from primary alcohols, secondary alcohols, long-hydrocarbon-chain alcohols and aromatic alcohols.

20. The composition according to claim 19, wherein the at least one substrate for the enzyme is present in an amount ranging from 0.01% to 60% by weight relative to the total weight of the composition.

21. The composition according to claim 1, wherein the at least one oxidation dye precursor is chosen from para-phenylenediamines, bis(phenyl)alkylenediamines, para-aminophenols, ortho-aminophenols and heterocyclic bases, and the addition salts thereof.

22. The composition according to claim 21, wherein the at least one oxidation dye precursor is present in an amount ranging from 0.0001% to 20% by weight relative to the total weight of the composition.

23. The composition according to claim 1, further comprising at least one coupler chosen from meta-phenylenediamines, meta-aminophenols, meta-diphenols, naphthalene-based couplers and heterocyclic couplers, and the addition salts thereof.

24. The composition according to claim 23, wherein the at least one coupler is present in an amount ranging from 0.0001% to 20% by weight relative to the total weight of the composition.

25. The composition according to claim 1, further comprising at least one direct dye chosen from cationic and natural direct dyes.

26. A process for dyeing keratin fibers, comprising applying to the keratin fibers at least one composition for a period of time sufficient to develop a desired coloration, wherein the at least one composition comprises, in a medium suitable for dyeing, at least one oxidation dye precursor; at least one alcohol oxidase enzyme; at least one substrate bearing at least one alcohol functional group for said enzyme; and at least one polymer comprising at least one sugar unit, wherein said at least one substrate may be totally or partially replaced by the at least one oxidation dye precursor in the case where the at least one oxidation dye precursor bears at least one alcohol functional group chosen from aromatic and aliphatic alcohol functional groups.

27. The process according to claim 26, wherein the at least one composition is a ready-to-use composition and is stored in anaerobic form, free of oxygen gas.

28. The process according to claim 27, further comprising a preliminary operation comprising separately storing, on the one hand, a composition (A) comprising, in a medium suitable for dyeing, the at least one oxidation dye precursor, and, on the other hand, a composition (B) comprising, in a medium suitable for dyeing, the at least one alcohol oxidase enzyme, wherein at least one of the composition (A) and the composition (B) comprises the at least one substrate for the enzyme and the at least one polymer comprising at least one sugar unit, and then mixing together the compositions (A) and (B) at the time of use before applying this mixture to the keratin fibers.

29. The process according to claim 28, further comprising a preliminary operation comprising separately storing, on the one hand, a composition (A) comprising, in a medium suitable for dyeing, the at least one oxidation dye precursor, the at least one substrate for the enzyme, and the at least one polymer comprising at least one sugar unit and, on the other hand, a composition (B) comprising, in a medium suitable for dyeing, the at least one alcohol oxidase enzyme, and then mixing together the compositions (A) and (B) at the time of use before applying this mixture to the keratin fibers.

30. A multi-compartment device, comprising
a first compartment comprising a composition (A) comprising, in a medium suitable for dyeing, at least one oxidation dye precursor, and
a second compartment comprising a composition (B) comprising, in a medium suitable for dyeing, at least one alcohol oxidase enzyme,
wherein at least one of the composition (A) and the composition (B) comprises at least one substrate bearing at least one alcohol functional group for the enzyme and at least one polymer comprising at least one sugar unit and
wherein said at least one substrate may be totally or partially replaced by the at least one oxidation dye precursor in the case where the at least one oxidation dye precursor bears at least one alcohol functional group chosen from aromatic and aliphatic alcohol functional groups.
